(19) United States
(12) Patent Application Publication
(10) Pub. No.: US 2022/0338733 A1
(43) Pub. Date: Oct. 27, 2022

(54) EXTERNAL ALIGNMENT INDICATION/GUIDANCE SYSTEM FOR RETINAL CAMERA

(71) Applicant: Verily Life Sciences LLC, South San Francisco, CA (US)

(72) Inventor: Ryan Kramer, San Francisco, CA (US)

(21) Appl. No.: 17/642,607

(22) PCT Filed: Sep. 23, 2020
(86) PCT No.: PCT/US2020/052194

§ 371 Date: Mar. 11, 2022

Related U.S. Application Data
(60) Provisional application No. 62/927,351, filed on Oct. 29, 2019.

(51) Int. Cl. A61B 3/15; A61B 3/107; A61B 3/00; A61B 3/113; A61B 3/12; A61B 3/11; A61B 3/18

(52) U.S. Cl. CPC A61B 3/152; A61B 3/107; A61B 3/0041; A61B 3/0008; A61B 3/113; A61B 3/0091; A61B 3/12; A61B 3/112; A61B 3/18

(57) ABSTRACT

A retinal camera system comprises an eyepiece lens disposed within a housing, a retinal image sensor, and a visual guidance indicator. The retinal image sensor is adapted to acquire a retinal image of an eye through the eyepiece lens. The visual guidance indicator is disposed in or on the housing peripherally about the eyepiece lens. The visual guidance indicator is positioned and oriented relative to the eyepiece lens to emit a visual cue along an optical path that does not pass through the eyepiece lens. The visual cue is adapted to facilitate alignment of the eye to the eyepiece lens.

### CROSS-REFERENCE TO RELATED APPLICATIONS

This application is based on U.S. Provisional Application No. 62/927,351, filed Oct. 29, 2019, the content of which is hereby incorporated by reference in its entirety.

### TECHNICAL FIELD

This disclosure relates generally to ophthalmic imaging technologies, and in particular but not exclusively, relates to alignment techniques for retinal imaging.

### BACKGROUND INFORMATION

Retinal imaging is a part of basic eye exams for screening, field diagnosis, and progress monitoring of many retinal diseases. A high fidelity retinal image is important for accurate screening, diagnosis, and monitoring. Bright illumination of the posterior interior surface of the eye (i.e., retina) through the pupil improves image fidelity but often creates optical aberrations or image artifacts, such as corneal reflections, iris reflections, or lens flare, if the retinal camera and illumination source are not adequately aligned with the eye. Simply increasing the brightness of the illumination does not overcome these problems, but rather makes the optical artifacts more pronounced, which undermines the goal of improving image fidelity.

Accordingly, camera alignment is very important, particularly with conventional retinal cameras, which typically have a very limited eyebox due to the need to block the deleterious image artifacts listed above. The eyebox for a retinal camera is a three dimensional region in space typically defined relative to an eyepiece of the retinal camera and within which the center of a pupil or cornea of the eye should reside to acquire an acceptable image of the retina. The small size of conventional eyeboxes makes retinal camera alignment difficult and patient interactions during the alignment process often strained.

Various solutions have been proposed to alleviate the alignment problem. For example, moving/motorized stages that automatically adjust the retina-camera alignment have been proposed. However, these stages tend to be mechanically complex and substantially drive up the cost of a retinal imaging platform. An effective and low cost solution for efficiently and easily achieving eyebox alignment of a retinal camera would improve the operation and market penetration of retinal cameras.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1 illustrates a retinal image including an demonstrative image artifact due to misalignment of the retinal camera.

FIG. 2 is a functional component diagram illustrating a retinal imaging system with an external visual guidance indicator for coarse alignment, in accordance with an embodiment of the disclosure.

FIG. 3A is a partial cross-sectional illustration of a visual guidance indicator disposed on the distal end of a lens tube about the eyepiece lens, in accordance with an embodiment of the disclosure.

FIG. 3B is an end view illustration of the visual guidance indicator including a single ring of emission locations disposed about the eyepiece lens, in accordance with an embodiment of the disclosure.

FIG. 4 is an end view illustration of a visual guidance indicator including concentric rings of emission locations disposed about the eyepiece lens, in accordance with an embodiment of the disclosure.

FIG. 5 is a flow chart illustrating a process of operation for a retinal camera system including an externally positioned visual guidance indicator for aiding coarse alignment, in accordance with an embodiment of the disclosure.

### DETAILED DESCRIPTION

Embodiments of a system, apparatus, and method for aligning an eyepiece lens of a retinal camera system to an eye are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

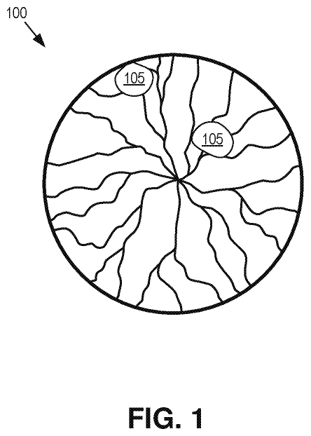

High fidelity retinal images are important for screening, diagnosing, and monitoring many retinal diseases. To this end, reducing or eliminating instances of image artifacts that occlude, or otherwise malign portions of the retinal image is desirable. FIG. 1 illustrates an example retinal image 100 with multiple image artifacts 105. These image artifacts may arise when misalignment between the retinal imaging system and the eye permit stray light and deleterious reflections from the illumination source to enter the imaging path and ultimately are captured by the retinal image sensor with the image light.

To capture a retinal image, the lens tube (including the eyepiece lens) must be precisely aligned with a subject's eye (usually to a tolerance of just a few millimeters). In order to achieve this precise alignment, most retinal cameras include some sort of fixation target in the optical path that is visible when looking directly into the eyepiece lens. Among other purposes, the fixation target provides feedback about where to look during alignment. However, due to the optical properties of typical lens tubes, even just getting one's eye to the region in space where the fixation target is visible is often challenging. Without any visual feedback to facilitate a coarse alignment, a grossly misaligned user is often unsure how to move relative to the eyepiece lens to gain visual contact with the fixation target, at which point fine or precise alignment can begin using the fixation target.

In order to assist the end user or patient in attaining an initial (coarse) alignment with the eyepiece lens, embodiments disclosed herein use a visual guidance indicator disposed in or on a distal end of the lens tube peripherally around the eyepiece lens. The visual guidance indicator emits a visual cue externally from the imaging path through the eyepiece lens (i.e., the visual cue does not pass through the eyepiece lens). The visual cue is adapted to guide the eye into sufficient coarse alignment with the eyepiece lens such that the user can see the fixation target, which is then used for fine alignment in preparation for obtaining a high fidelity retinal image. The fine alignment may be achieved via high precision retinal tracking through the eyepiece lens using the retinal image sensor itself.

In some embodiments, the pupil or iris is used to coarsely track the relative position of the eye to the eyepiece lens. This coarse tracking system may then be used to dynamically alter the visual cue to provide real-time guidance feedback to the user's eye. The dynamic changes may include changes in brightness of the visual cue, changes in a shape-pattern of the visual cue, changes in a temporal-pattern of the visual cue, changes in colors of the visual cue, or combinations thereof. These dynamic changes may provide an intuitive visual feedback guidance system to aid a user in the initial coarse/gross alignment. Embodiments described herein enable fully automated retinal camera systems that can be used without a skilled technician's intervention, thereby opening up a variety of new uses cases and environments of operation.

Finally, the visual guidance indicator along with the external alignment tracking camera (e.g., pupil or iris tracking system) may be further leveraged to provide additional screen and diagnostic testing. For example, the visual guidance indicator may provide exterior illumination for anterior segment imaging, pupillometry to measure the pupil size as well as pupillary light reflex (PLR), and/or three-dimensional (3D) surface topography (e.g., measuring the anterior surface curvature of the cornea), which is conventionally performed by a Keratometer—not retinal camera systems.

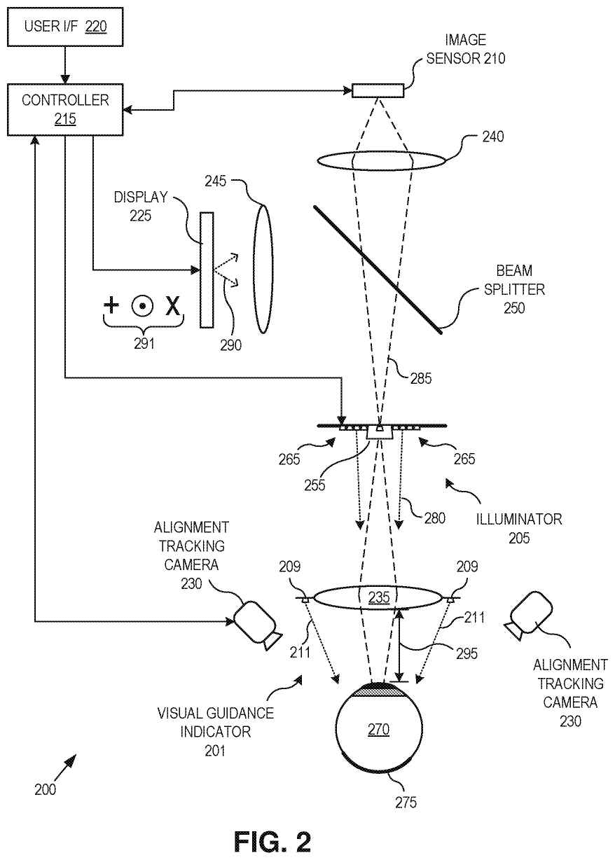

FIG. 2 illustrates a retinal imaging system 200 with an external visual guidance indicator, in accordance with an embodiment of the disclosure. The illustrated embodiment of retinal imaging system 200 includes a visual guidance indicator 201, an illuminator 205, an image sensor 210 (also referred to as a retinal image sensor), a controller 215, a user interface 220, a display 225, alignment tracking camera(s) 230, and an optical relay system. The illustrated embodiment of the optical relay system includes lens assemblies 235, 240, 245 and a beam splitter 250. The illustrated embodiment of illuminator 205 comprises illuminator arrays 265 and a center aperture 255. The illustrated embodiment of visual guidance indicator 201 including emission locations 209.

The optical relay system serves to direct (e.g., pass or reflect) illumination light 280 output from illuminator 205 along an illumination path through the pupil of eye 270 to illuminate retina 275 while also directing image light 285 of retina 275 (i.e., the retinal image) along an imaging path to image sensor 210. Image light 285 is formed by the scattered reflection of illumination light 280 off of retina 275. In the illustrated embodiment, the optical relay system further includes beam splitter 250, which passes at least a portion of image light 285 to image sensor 210 while also optically coupling fixation target 291 to eyepiece lens assembly 235 and directing display light 290 output from display 225 to eye 270. Beam splitter 250 may be implemented as a polarized beam splitter, a non-polarized beam splitter (e.g., 90% transmissive and 10% reflective, 50/50 beam splitter, etc.), a dichroic beam splitter, or otherwise. The optical relay system includes a number of lenses, such as lenses 235, 240, and 245, to focus the various light paths as needed. For example, lens 235 may include one or more lensing elements that collectively form an eyepiece lens that is housed within a lens tube (not illustrated in FIG. 1). The eyepiece lens is displaced from the cornea of eye 270 by an eye relief 295 during operation. Lens 240 may include one or more lens elements for bringing image light 285 to a focus on image sensor 210. Lens 245 may include one or more lens elements for focusing display light 290. It should be appreciated that optical relay system may be implemented with a number and variety of optical elements (e.g., refractive lenses, reflective surfaces, diffractive surfaces, etc.) and may vary from the configuration illustrated in FIG. 2.

In one embodiment, display light 290 output from display 225 represents a fixation target. The fixation target may be an image of a plus-sign, a bullseye, a cross, a target, or other shape (e.g., see demonstrative fixation target images 291). The fixation target not only can aid with obtaining fine or precise alignment between eyepiece lens 235 and eye 270 by providing visual feedback to the patient, but also gives the patient a fixation target upon which to accommodate and stabilize their vision. Display 225 may be implemented with a variety of technologies including an liquid crystal display (LCD), light emitting diodes (LEDs), various illuminated shapes (e.g., an illuminated cross or concentric circles), or otherwise. Of course, the fixation target may be implemented in other manners than a virtual image on a display. For example, the fixation target may be a physical object (e.g., crosshairs, etc.).

The illustrated embodiment of visual guidance indicator 201 is disposed in or on the housing surrounding eyepiece lens 235. This housing may be a lens tube that incorporates eyepiece lens 235. Visual guidance indicator 201 includes emission locations 209 for outputting visual cue 211 externally from the imaging path through eyepiece lens 325. In other words, visual guidance indicator 201 is positioned and oriented relative to eyepiece lens 235 to emit visual cue 211 to eye 270 along an optical path to eye 270 that does not pass through eyepiece lens 235. Visual guidance indicator 201 is positioned on the distal end of the lens tube and peripherally surrounds eyepiece lens 235 to directly and externally present eye 270 with its visual cue 211.

Image sensor 210 may be implemented using a variety of imaging technologies, such as complementary metal-oxide-semiconductor (CMOS) image sensors, charged-coupled device (CCD) image sensors, or otherwise. In one embodiment, image sensor 210 includes an onboard memory buffer or attached memory to store/buffer retinal images.

Alignment tracking camera(s) 230 operate to track lateral and eye relief offset alignment (or misalignment) between retinal imaging system 200 and eye 270, and in particular, between eyepiece lens assembly 235 and eye 270. Alignment tracking camera 230 may operate using a variety of different techniques to track the relative position of eye 270 to retinal imaging system 200 including pupil tracking, iris tracking, or otherwise. In the illustrated embodiment, alignment tracking camera 230 includes two cameras disposed on either side of eyepiece lens assembly 235 to enable triangulation and obtain X, Y, and Z position information about the pupil or iris. In one embodiment, alignment tracking camera 230 includes one or more infrared (IR) emitters to track eye 270 via IR light while retinal images are acquired with visible spectrum light, and in some cases, with IR light as well.

Eye position, including lateral alignment and/or eye relief offset alignment, may be measured and tracked using retinal images acquired by image sensor 210 for precise alignment tracking, or separately/additionally, by alignment tracking camera(s) 230. Alignment tracking camera(s) 230 provide coarse alignment tracking via the pupil or iris. In the illustrated embodiment, alignment tracking camera(s) 230 are positioned externally to view eye 270 from outside of eyepiece lens assembly 235. In other embodiments, alignment tracking camera(s) 230 may be optically coupled via the optical relay components to view and track eye 270 through eyepiece lens assembly 235.

Controller 215 is coupled to image sensor 210, display 225, illuminator 205, alignment tracking camera 230, and visual guidance indicator 201 to choreograph their operation. Controller 215 may include software/firmware logic executing on a microcontroller, hardware logic (e.g., application specific integrated circuit, field programmable gate array, etc.), or a combination of software and hardware logic. Although FIG. 2 illustrates controller 215 as a distinct functional element, the logical functions performed by controller 215 may be decentralized across a number hardware elements. Controller 115 may further include input/output (I/O ports), communication systems, or otherwise. Controller 215 is coupled to user interface 220 to receive user input and provide user control over retinal imaging system 200. User interface 220 may include one or more buttons, dials, feedback displays, indicator lights, etc.

During operation, controller 115 operates illuminator 205 and retinal image sensor 210 to capture one or more retinal images. Illumination light 280 is directed through the pupil of eye 270 to illuminate retina 275. The scattered reflections from retina 275 are directed back along the image path through aperture 255 to image sensor 210. When eye 270 is properly aligned within the eyebox of system 200, aperture 255 operates to block deleterious reflections and light scattering that would otherwise malign the retinal image while passing the image light itself. Prior to capturing the retinal image, controller 215 operates visual guidance indicator 201 and alignment tracking camera(s) 230 to provide real-time visual feedback (i.e., visual cue 211) to eye 270 to achieve coarse alignment, at which point the user can see the fixation target. Controller 215 further operates display 225 to output a fixation target image 291 to guide the patient's gaze into fine or precise alignment. Once fine alignment is achieved, controller 215 deems eye 270 to be within the eyebox of retinal imaging system 200, and thus acquires a retinal image with image sensor 210.

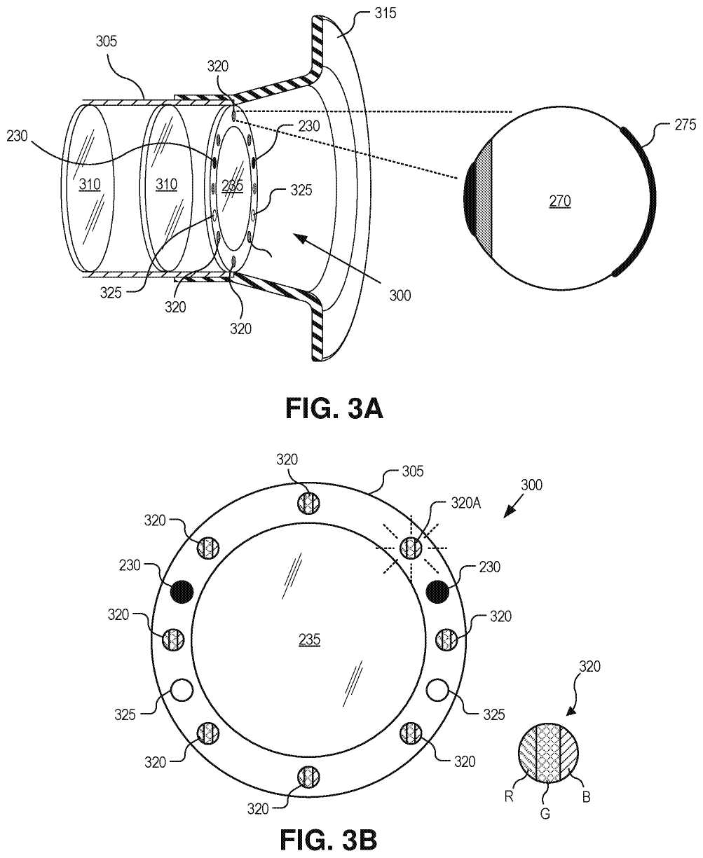

FIGS. 3A and 3B illustrate an example visual guidance indicator 300, in accordance with an embodiment of the disclosure. FIG. 3A is a partial cross-sectional illustration of visual guidance indicator 300 while FIG. 3B is a frontal view illustration of the same. Visual guidance indicator 300 is disposed on the distal end of a housing 305 encasing eyepiece lens 235. In the illustrated embodiment, housing 305 is a lens tube that holds eyepiece lens 235 along with one or more additional lens elements 310 for focusing or magnifying image light. An eyecup 315 (only illustrated in FIG. 3A) may also mount to the distal end of housing 305 to help position eye 275 relative to eyepiece lens 235, block out stray ambient light to control ambient lighting, and provide a soft, comfortable surface against which the patient may press their eye socket. In other embodiments, eyecup 315 may be replaced with a facemask that fits around both eyes. In the illustrated embodiment, visual guidance indicator 300 is disposed within eyecup 315, which encircles visual guidance indicator 300.

As illustrated, visual guidance indicator 300 includes a number of emission locations 320 disposed on the distal exterior end of housing 305. Emission locations 320 face outward to directly present eye 270 with their visual cue. Emission locations 320 peripherally surround eyepiece lens 235. In the illustrated embodiment, emission locations 320 are radially outside eyepiece lens 235, but within eyecup 315. The distal end of housing 305 further includes alignment tracking cameras 230 and IR illuminators 325.

In the illustrated embodiment, each emission location 320 includes a multi-color illumination source, such as a set of red (R), green (G), and blue (B) light emitting diodes (LEDs). Of course, other color combinations with more or less color LEDs may be implemented. Emission locations 320 may be operated to provide various patterns, shapes, colors, or intensities to aid eye alignment. For example, emission locations 320 may form a ring centered around eyepiece lens 235. The ring may provide a visual target within which the user centers eye 270. Visual guidance indicator 300 may dynamically alter the visual cue output from emission locations 320 to provide real-time feedback guidance to eye 270. One or more of a brightness, a shape-pattern, a temporal-pattern, or colors of the visual cue output by visual guidance indicator 300 may be dynamically altered to provide intuitive feedback. For example, emission locations 320 may initially glow a first color (e.g., red) to provide the user with an alignment reference. As the user's eye 270 approaches eyepiece lens 235, various emission locations 320 may blink to indicate a directional adjustment. As an example, in FIG. 3B emission location 320A is blinking to indicate that the user needs to adjust their eye up and to the right. Of course, other emission locations 320 may blink, change color, or change intensity to indicate other directional instructions. In one embodiment, as eye 270 approaches coarse alignment (e.g., alignment sufficient to see fixation target image 291 through eyepiece lens 235), the visual cue output from visual guidance indicator 300 may start to dim or even go dark so as not to distract eye 270 from the precise alignment target provided by fixation target image 291 and not interfere with retinal imaging. If eye 270 loses its coarse alignment, then visual guidance indicator 300 may resume illumination to provide coarse alignment feedback again.

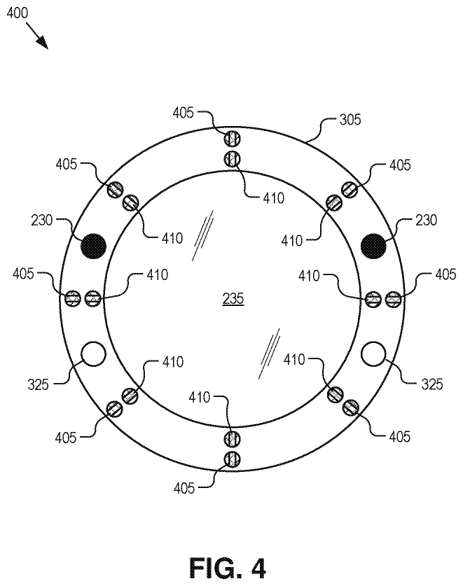

FIGS. 3A and 3B illustrate an embodiment of visual guidance indicator 300 that includes only a single ring of emission sources 320. FIG. 4 is an end view illustration of a visual guidance indicator 400 with multiple illumination rings, which represents another possible implementation of visual guidance indicator 201. Visual guidance indicator 400 includes a two concentric rings of emission locations 405 and 410 disposed on the distal end of housing 305 and extend peripherally around eyepiece lens 235. Emission locations 405 and 410 may be implemented similar to emission locations 320 (e.g., RGB LEDs, monochrome sources, light pipes, light rings, or otherwise). However, the inclusion of multiple rings having different radial offsets from center enables the generation of the visual cue with more complex shape-patterns and temporal-patterns to provide the user with more informative feedback.

Additionally, visual guidance indicator 400 (also 300 or 201) may be used to provide general exterior ambient illumination for imaging other aspects of eye 270 than just retina 275. For example, these visual guidance indicators may provide exterior illumination for anterior segment imaging (e.g., imaging of the cornea, iris, sclera, eyelid, lashes, tear duct, etc.), pupillometry testing, or 3D topographical mapping of the cornea. While these additional uses for the visual guidance indicators can be implemented with the single ring embodiment illustrated in FIGS. 3A and 3B, the multi-ring embodiment of FIG. 4 facilitates greater flexibility with this imaging. For example, corneal topography and/or photometric stereo imaging of the cornea seek to map the curvature of the cornea by observing the cornea under different lighting conditions or using different illumination reference patterns. These different light conditions may be achieved by visual guidance indicator 300 by simply adjusting the eye relief position of eyepiece lens 235 (e.g., moving the retinal imaging system closer to eye 270). Alternatively, visual guidance indicator 400 may be held at a constant eye relief position, and the incident angles of illumination adjusted using the different illumination rings.

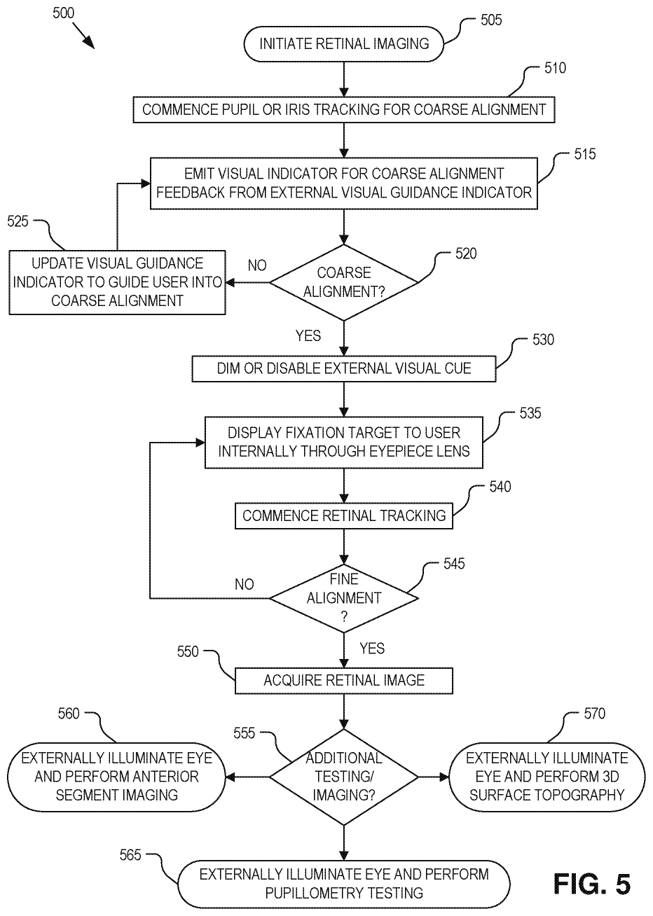

FIG. 5 is a flow chart illustrating a process 500 of operation for retinal imaging system 200 including an externally positioned visual guidance system 201 (also 300 or 400) for aiding coarse alignment, in accordance with an embodiment of the disclosure. The order in which some or all of the process blocks appear in process 500 should not be deemed limiting. Rather, one of ordinary skill in the art having the benefit of the present disclosure will understand that some of the process blocks may be executed in a variety of orders not illustrated, or even in parallel.

In a process block 505, the imaging process is initiated. Initiation may begin when the patient's eye is placed in front of eyepiece lens 235 and/or upon selection of an initiation command (e.g., start button or capture button). In a process block 510, controller 215 begins monitoring the patient's eye alignment. As mentioned above, coarse eye alignment may be determined via alignment tracking camera(s) 230, which may perform pupil or iris tracking for gross eye alignment.

As part of the coarse alignment procedure, visual guidance indicator 201 is enabled emitting visual cue 211, which is adapted to facilitate alignment of eye 270 to eyepiece lens 235. For example, initially a ring of emission locations 320 may glow an initial color (e.g., red) to provide eye 270 with a reference for alignment. Controller 215 may then use alignment tracking camera(s) 230 to track/monitor the relative coarse alignment. If coarse alignment has not yet been achieved (decision block 520), then controller 215 may use visual guidance indicator 201 to provide visual feedback guidance cues (process block 525). Visual cue 211 may be dynamically altered in real-time to provide dynamic feedback guidance based upon the pupil or iris tracking to encourage coarse alignment. For example, various emission locations 320 may blink, change color, or change intensity (or temporal/spatial combinations thereof) to direct eye 270 in the particular lateral or eye relief direction needed to achieve coarse alignment. As eye 270 achieves coarse alignment (decision block 520), visual cue 211 may change color (e.g., change to green) to provide a visual confirmation of coarse alignment and the brightness of the visual cue may also be dimmed or disabled (process block 530) as a sort of alignment handoff from the visual guidance indicator 201 to fixation target 291. Visual cue 211 may also be dimmed or disabled prior to acquiring the retinal image so as not to introduce deleterious reflections.

In a process block 535, a fixation target is internally displayed to eye 270 through eyepiece lens 235. As previously mentioned, the fixation target not only provides a fixation location to steady the user's gaze during precise or fine alignment, but also helps the user accommodate the optical power of their vision to the correct focal distance. With the user's vision fixated on the fixation target and eye 270 coarsely aligned, controller 215 can perform retinal tracking (process block 540) using image sensor 210 to determine fine alignment (decision block 545). Retinal tracking uses retinal images acquired by image sensor 210 through eyepiece lens 235 that are analyzed by controller 215 to determine alignment. The retinal images used for alignment are pre-alignment images that may have significant artifacts until precise alignment is achieved (i.e., eye 270 is aligned into the eyebox of retinal imaging system 200). Once fine alignment is achieved within threshold tolerances, and/or for a threshold period of time, one or more retinal images are acquired (process block 550).

In addition to retinal imaging, retinal camera system 200 with visual guidance indicator 201 may be used to perform a variety of other ophthalmic diagnostic imaging/testing (decision block 555). Visual guidance indicator 201 along with alignment tracking camera(s) 230 enables retinal camera system 200 to be leveraged for additional imaging/test beyond just acquiring a retinal image. For example, visual guidance indicator 201 may be used as an ambient/external illumination source to perform one or more of the following: anterior segment imaging (process block 560), pupillometry testing (process block 565), or 3D surface topography of the cornea (process block 570). In other words, visual guidance indicator 201 may be used not just as a visual cue to guide eye 270 into gross alignment, but also to provide ambient illumination to achieve appropriate exposure levels for the additional imaging/testing. Although FIG. 5 illustrates this additional imaging as occurring after acquisition of a retinal image, the additional testing/imaging may occur prior to retinal imaging, while attempting to acquire coarse alignment, immediately after achieving coarse alignment but prior to fine alignment, while seeking fine alignment, entirely independent of retinal imaging or otherwise.

Anterior segment imaging (process block 560) includes imaging one or more of the cornea, the iris, the sclera, an eyelid, an eyelash, or a tear duct. During anterior segment imaging, the visual guidance indicator 201 may enable visual guidance indicator 201 to provide visible white light illumination. In one embodiment, white light illumination may be achieved by simultaneously flashing the R, G, and B LEDs at each emission location 320. The anterior segment images may be acquired by one or more of alignment tracking camera(s) 230 and/or image sensor 210. Additionally, visual guidance indicator 201 may be used to guide eye 270 to the appropriate lateral and/or eye relief location for the anterior segment imaging. This position may not be the same position as for retinal imaging.

Pupillometry testing (process block 565) involves measuring the size of a pupil, as well as, the pupillary light reflex (PLR). PLR is the pupillary response to a light stimulus. Having visual guidance indicator 201 directly facing eye 270 on the exterior distal end of retinal camera system 200 enables varying the light levels or intensities output from emission locations 320 and recording the pupillary response. Either image sensor 210 or alignment tracking camera(s) 230 may be used to record the pupillary response. Visual guidance indicator 201 may be operated to emit white light illumination (simultaneously enabling RGB LEDs) or any selected color individually to measure color specific PLR.

3D surface topography (process block 570) involves measuring and/or mapping the 3D surface of the cornea. Conventionally, 3D surface topography requires the use of a dedicated machine such as a Keratometer; however, embodiments described herein leverage visual guidance indicator 201 to this end. 3D surface topography may be performed using a variety of techniques. In one embodiment, a photometric stereo imaging technique is performed by rapidly and sequentially illuminating the various emission locations 320 to observe the corneal reflections under different lighting conditions. By analyzing the reflections, this technique allows for the estimation of corneal surface normals via ray tracing from multiple different source locations to determine the surface curvature of the cornea. In yet another embodiment, a corneal topograph may be acquired by using the various emission locations of visual guidance indicator 201 to illuminate the cornea with one or more known reference patterns and record the corneal reflections. These reflections can then be analyzed (e.g., compared against reference reflection patterns) to determine the surface curvature. Again, the reflections may be captured with alignment tracking camera(s) 230 and/or image sensor 210

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A retinal camera system, comprising:
a housing;
an eyepiece lens disposed within the housing;
a retinal image sensor optically coupled to the eyepiece lens to acquire a retinal image of an eye through the eyepiece lens; and
a visual guidance indicator disposed in or on the housing peripherally about the eyepiece lens, the visual guidance indicator positioned and oriented relative to the eyepiece lens to emit a visual cue along an optical path that does not pass through the eyepiece lens, the visual cue adapted to facilitate alignment of the eye to the eyepiece lens.

2. The retinal camera system of claim 1, wherein the visual guidance indicator comprises a plurality of emission locations disposed about the eyepiece lens from which the eye can reference alignment with the eyepiece lens.

3. The retinal camera system of claim 2, wherein the plurality of emission locations form two concentric rings extending around the eyepiece lens.

4. The retinal camera system of claim 1, wherein the housing comprises a lens tube including the eyepiece lens and wherein the visual guidance indicator is disposed on a distal exterior end of the lens tube and facing outwards to present the eye with the visual cue.

5. The retinal camera system of claim 1, further comprising a controller communicatively coupled to the visual guidance indicator and the retinal image sensor, the controller including logic that when executed by the controller causes the ophthalmic camera system to perform operations including:
dynamically altering one or more of a brightness of the visual cue, a shape-pattern of the visual cue, a temporal-pattern of the visual cue, or colors of the visual cue to aid alignment of the eye to the eyepiece lens.

6. The retinal camera system of claim 5, wherein the controller includes further logic that when executed by the controller causes the ophthalmic camera system to perform additional operations including:
emitting the visual cue to aid a coarse alignment of the eye to the eyepiece lens; and
dimming or disabling the visual cue in advance of acquiring the retinal image of the eye.

7. The retinal camera system of claim 5, further comprising:
a display communicatively coupled to the controller, the display optically coupled to the eyepiece lens to emit a fixation target to the eye through the eyepiece lens, wherein the visual guidance indicator is adapted to facilitate a coarse alignment between the eye and the eyepiece lens to guide the eye into sufficient alignment to see the fixation target and the fixation target is adapted to then facilitate a fine alignment between the eye and the eyepiece lens for retinal imaging with the retinal image sensor.

8. The retinal camera system of claim 5, further comprising:
an alignment tracking camera communicatively coupled to the controller, disposed peripherally to the eyepiece lens, and positioned to provide pupil or iris tracking of the eye,
wherein the controller includes further logic that when executed by the controller causes the ophthalmic camera system to perform additional operations including:
tracking a relative position of the eye to the eyepiece lens;
dynamically altering the visual cue based on the tracking to provide feedback guidance to the eye for achieving the coarse alignment, wherein the feedback guidance includes cues visually instructing a user to move the eye relative to the eyepiece lens in a lateral direction or an eye relief direction.

9. The retinal camera system of claim 8, wherein the controller includes further logic that when executed by the controller causes the ophthalmic camera system to perform additional operations including:
illuminating the eye with the visual guidance indicator; and
acquiring an anterior segment image of the eye with at least one of the alignment tracking camera or the retinal image sensor while using the visual guidance indicator to provide illumination for the anterior segment image.

10. The retinal camera system of claim 8, wherein the controller includes further logic that when executed by the controller causes the ophthalmic camera system to perform additional operations including:
illuminating the eye with the visual guidance indicator;
varying an intensity of the illuminating; and
measuring pupillary reactions to the varying of the intensity with at least one of the alignment tracking camera or the retinal image sensor to perform pupillometry testing of a pupil of the eye.

11. The retinal camera system of claim 8, wherein the controller includes further logic that when executed by the controller causes the ophthalmic camera system to perform additional operations including:
sequentially activating different angular positions of the visual guidance indicator about the eyepiece lens to illuminate the eye from the different angular positions;
observing reflections off a cornea of the eye with at least one of the alignment tracking camera or the retinal image sensor; and
determining a surface curvature of the cornea based upon the reflections.

12. The retinal camera system of claim 8, wherein the controller includes further logic that when executed by the controller causes the ophthalmic camera system to perform additional operations including:
activating the visual guidance indicator to illuminate the eye with a reference pattern;
capturing a reflection of the reference pattern off of a cornea of the eye with at least one of the alignment tracking camera or the retinal image sensor; and
analyzing the reflection to determine a surface curvature of the cornea.

13. A method for imaging an eye, the method comprising:
emitting a fixation target through an eyepiece lens towards the eye, wherein the fixation target facilitates a fine alignment between the eye and the eyepiece lens;
emitting a visual cue from a visual guidance indicator surrounding an exterior side of the eyepiece lens, wherein the visual cue does not pass through the eyepiece lens and provides a visual reference to guide the eye into a coarse alignment with the eyepiece lens to observe the fixation target; and
capturing a retinal image of the eye through the eyepiece lens with a retinal image sensor.

14. The method of claim 13, further comprising:
dynamically altering one or more of a brightness of the visual cue, a shape-pattern of the visual cue, a temporal-pattern of the visual cue, or colors of the visual cue to guide the eye into the coarse alignment.

15. The method of claim 13, further comprising:
dimming or disabling the visual cue after the eye achieves the coarse alignment and prior to capturing the retinal image.

16. The method of claim 13, further comprising:
tracking a pupil or an iris of the eye with an alignment tracking camera;
determining a relative position of the eye to the eyepiece lens based upon the tracking;
dynamically altering the visual cue based on the relative position to provide feedback guidance to the eye for achieving the coarse alignment, wherein the feedback guidance includes cues visually instructing a user to move the eye relative to the eyepiece lens in a lateral direction or an eye relief direction.

17. The method of claim 16, further comprising:
illuminating the eye with the visual guidance indicator; and
acquiring an anterior segment image of the eye with at least one of the alignment tracking camera or the retinal image sensor while using the visual guidance indicator to provide illumination for the anterior segment image.

18. The method of claim 16, further comprising:
illuminating the eye with the visual guidance indicator;
varying an intensity of the illuminating; and
measuring pupillary reactions to the varying of the intensity with at least one of the alignment tracking camera or the retinal image sensor to perform pupillometry testing of a pupil of the eye.

19. The method of claim 16, further comprising:
sequentially activating different angular positions of the visual guidance indicator about the eyepiece lens to illuminate the eye from the different angular positions;
observing reflections off a cornea of the eye with at least one of the alignment tracking camera or the retinal image sensor; and
determining a surface curvature of the cornea based upon the reflections.

20. The method of claim 16, further comprising:
activating the visual guidance indicator to illuminate the eye with a reference pattern;
capturing a reflection of the reference pattern off of a cornea of the eye with at least one of the alignment tracking camera or the retinal image sensor; and
analyzing the reflection to determine a surface curvature of the cornea.
